SEQUENCE LISTING (6 sequences)

SEQ ID NO: 1 - DNA, 12 bp, Artificial sequence
  Uni-12 primer
agcaaaagcagg

SEQ ID NO: 2 - DNA, 29 bp, Artificial sequence
  H1N1 504F primer
agtttttacaraaatttgctatggctgac

SEQ ID NO: 3 - DNA, 27 bp, Artificial sequence
  H1N1 688R primer
ggggtgaatctyctgytataatktcaa

SEQ ID NO: 4 - DNA, 21 bp, Artificial sequence
  FluA-M52C primer
cttctaaccgaggtcgaaacg

SEQ ID NO: 5 - DNA, 24 bp, Artificial sequence
  FluA-M253R primer
agggcattttggacaaakcgtcta

SEQ ID NO: 6 - DNA, 21 bp, Artificial sequence
  M-fragment Probe
cctcaaagccgagatcgcgca

(19) United States
(12) Patent Application Publication
(10) Pub. No.: US 2013/0004939 A1
(43) Pub. Date: Jan. 3, 2013

(54) Fetuin-beads, Manufacturing method thereof and method of concentrating and detecting influenza virus by the same

(75) Inventors: Chung-Ming Chang, Taoyuan County (TW); Chiuan-Chian Chiou, Taoyuan County (TW)

(73) Assignee: Chang-Gung University, Taoyuan County (TW)

(21) Appl. No.: 13/135,280

(22) Filed: Jun. 30, 2011

(51) Int. Cl. C12Q 1/70

(52) U.S. Cl. 435/5

(57) ABSTRACT

Fetuin-beads, a manufacturing method thereof and a method of concentrating and detecting influenza virus by the fetuin-beads are disclosed. The method of concentrating and detecting an influenza virus by the fetuin-beads comprises the steps of preparing fetuin-beads; mixing the fetuin-beads and a solution capable of providing salt ions to obtain a fetuin-bead solution; mixing the fetuin-bead solution and a sample comprising the influenza virus to concentrate the influenza virus onto the fetuin-beads to obtain a plurality of fetuin bead-influenza virus combinations; and collecting the fetuin bead-influenza virus combinations and using a virus testing method to detect the influenza virus on the fetuin bead-influenza virus combinations. Therefore, an easy and fast method to detect the influenza virus with timeliness and high accuracy is provided.

### FIELD OF THE INVENTION

The present invention relates to a method of detecting influenza virus, in particular to fetuin-beads, a manufacturing method thereof and a method of concentrating and detecting influenza virus by the same. The method of concentrating and detecting influenza virus by the fetuin-beads is that virus particles are adsorbed and recovered onto the surfaces of the fetuin-beads by the magnetism of the beads so as to perform the influenza virus detection.

### BACKGROUND OF THE INVENTION

Bird flu continues spreading over the world, causing a considerable impact on human health and the poultry industry, so that timely detections and diagnostics are very important. The ways of obtaining a sample for detecting an influenza virus include: hunting wild waterfowl or making traps. However, in many countries, which include Taiwan, hunting wild waterfowl is not allowed. The present ways of obtaining the sample incur a higher level of difficulty because of the habits or alertness of the wild waterfowls to affect the sample collection results. Additionally, the sample obtained from some wild birds with a too-small size is also difficult to collect.

In addition, the launch of available medical clinical test reagents for a rapid influenza screening has the issue of a low sensitivity to the influenza virus in a clinical respiratory tract sample. Therefore, a fast screening detection of the influenza virus still has limitations on the accuracy of a physician's diagnosis and the timeliness of the dosage. Besides, a further culture of the sample is often required and a real-time quantitative polymerase chain (PCR) reaction is carried out in a laboratory to improve the chance of a highly accurate diagnosis. On the other hand, the timeliness of the detection is affected significantly. The main reason of a relatively low sensitivity for the above influenza virus detection resides on an insufficient concentration of the influenza virus in the original sample.

In summary, the conventional influenza virus detection system still has the shortcomings of a difficult sampling and an insufficient concentration of the influenza virus in collected samples to result in a poor accuracy on the influenza virus detection, such that feasible solutions are required.

### SUMMARY OF THE INVENTION

In view of the shortcomings of the conventional influenza virus detection with a difficult sample collection, a low sensitivity and a low accuracy of detecting an influenza virus, it is a primary objective of the present invention to overcome the shortcomings of the prior art by providing a method of concentrating and detecting influenza virus by fetuin-beads in accordance with the present invention.

To achieve the aforementioned objective, the present invention provides a manufacturing method of a fetuin-bead, comprising the following steps. A bead solution including a plurality of beads is prepared. A fetuin solution and a solution capable of providing salt ions are added into the bead solution to perform a shaking process and a culturing process to further obtain a fetuin-bead solution containing the plurality of fetuin-beads.

Preferably, a concentration of beads in the bead solution is in a range of 1×10⁹ to 4×10⁹/mL.

Preferably, the solution capable of providing salt ions is an ammonium sulfate solution.

Preferably the ammonium sulfate solution has a final concentration falling within a range of 2 M to 3 M.

Preferably, in the step of adding the fetuin solution and the solution capable of providing salt ions into the bead solution, the culturing process is taken place at 35° C. to 42° C. for 18 hours to 24 hours.

Preferably, the fetuin-bead solution has a content of fetuin-beads falling within a range of 40 to 50 μg.

The present invention further provides a fetuin-bead manufactured by the aforementioned manufacturing method of a fetuin-bead.

The present invention further provides a method of concentrating and detecting influenza virus by the fetuin-bead, comprising the following steps. The aforementioned fetuin-bead is provided. The fetuin-bead is mixed with a solution capable of providing salt ions uniformly to obtain a fetuin-bead solution. The fetuin-bead solution is mixed with a sample containing an influenza virus uniformly to concentrate the influenza virus onto the fetuin-bead to form a plurality of fetuin-bead influenza virus combinations. The fetuin-bead influenza virus combinations are collected after a specific time, and a virus detection method is used to detect the influenza virus concentrated in the fetuin-bead influenza virus combinations.

Preferably, the solution capable of providing salt ions is an ammonium sulfate solution.

Preferably, the ammonium sulfate solution has a final concentration falling within a range of 2 M to 3 M.

Preferably, the fetuin-bead solution used in the step of mixing the fetuin-bead solution and the sample containing the influenza virus has 2×10⁷ to 4×10⁷ fetuin-beads.

Preferably, the virus detection method is one selected from RNA extraction and real-time quantitative PCR, immunoblotting, and hemagglutination inhibition.

The technical measures taken by the fetuin-bead, the manufacturing method thereof and the method of concentrating and detecting influenza virus by the fetuin-beads in accordance with the present invention have the following advantages and effects.

(1) The fetuin-bead of the present invention can be applied for the recovery of influenza viruses in a natural water source with large-volume so as to detect and separate the influenza viruses rapidly, and used extensively in hospitals or research institutions, particularly for the applications on monitoring influenza viruses at early-stage identifications and preventing new influenzas.

(2) The method of concentrating and detecting influenza viruses by the fetuin-bead in accordance with the present invention can be applied in a water source with large-volume, so that the difficult sample collection of the feces of wild birds and the legal issue of capturing wild birds can be overcome. The method of concentrating and detecting the influenza viruses of the present invention are easy and quick influenza virus detection method, and comes with the timeliness feature and prevents possible pandemic influenzas.

(3) The fetuin-bead of the present invention is capable of concentrating the influenza virus effectively and surely, and the influenza virus concentrated by the fetuin-bead of the present invention still has the ability of infecting chicken embryos. This feature can be applied to subsequent subcultures including research of viruses to improve the clinical and future molecular biological analysis substantially.

### BRIEF DESCRIPTION OF THE DRAWINGS

FIG. 1 is a flow chart of a manufacturing method of a fetuin-bead in accordance with the present invention;

FIG. 2 is a flow chart of a method of concentrating and detecting influenza virus by the fetuin-bead in accordance with the present invention;

FIG. 3 is a SDS-PAGE diagram, showing the combination effect of different concentrations of fetuins combined with beads in accordance with the present invention;

FIG. 4 is a SDS-PAGE diagram, showing the combination effect of fetuins added with different concentrations of ammonium sulfate solution and combined with beads in accordance with the present invention;

FIG. 5 is a SDS-PAGE diagram, showing the combination effect of fetuins and beads measured at different heating temperatures in accordance with the present invention; and

FIG. 6 is a SDS-PAGE diagram, showing the optimally combination effect of fetuins and beads in accordance with the present invention.

### DESCRIPTION OF THE PREFERRED EMBODIMENTS

The following preferred embodiments are described in details for the purpose of illustrating the present invention, but not intended for limiting the scope of the present invention.

The fetuin of the present invention is a bovine serum albumin, and studies confirmed that the structure of fetuin has the structure of sialic acids α2,3 and α2,6 (as described by Baenziger and Fiete, Structure of the complex oligosaccharides of fetuin, J Biol Chem 254: 789-795, 1979). Influenza viruses are combined with the structure of sialic acids α2,3 and α2,6 of the fetuin through a surface antigen (hemagglutinin, HA) to infect cells (as described by Matrosovich et al., The surface glycoproteins of H5 influenza viruses isolated from humans, chickens, and wild aquatic birds have distinguishable properties, J Virol 73: 1146-1155, 1999). The present invention can increase the accuracy by means of the aforementioned binding property and using fetuin as an intermediate for adsorbing the influenza viruses.

### Embodiment 1

### Manufacturing Method of Fetuin-Beads

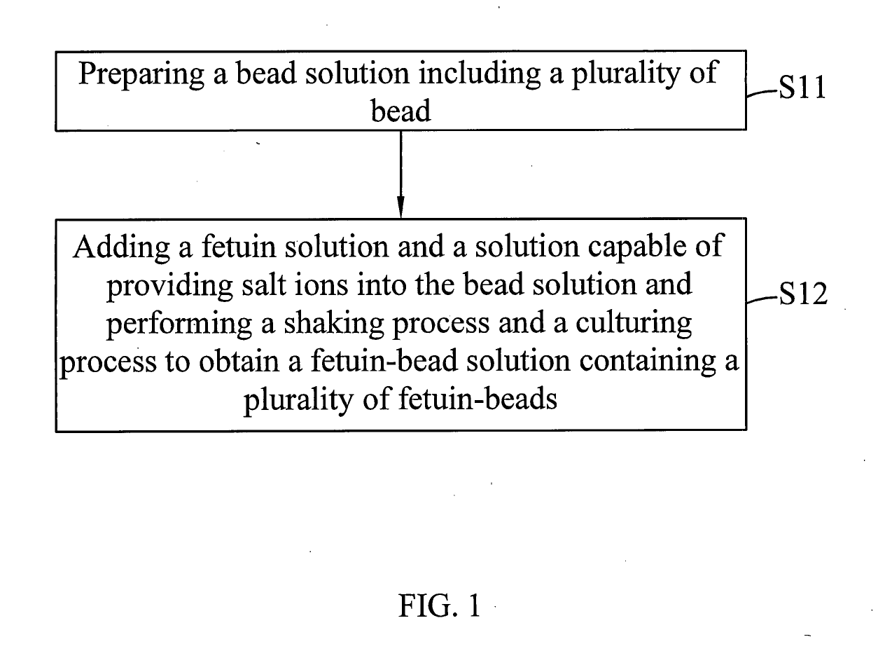

With reference to FIG. 1 for a flow chart of a manufacturing method of a fetuin-bead in accordance with the present invention, the manufacturing method comprises the following steps. In Step S11, a bead solution is prepared, wherein the bead solution comprises a plurality of beads. In Step S12, a fetuin solution and a solution capable of providing salt ions are added into the bead solution, and shaken and cultured to obtain a fetuin-bead solution, wherein the fetuin-bead solution comprises a plurality of fetuin-beads.

Preferably, the bead solution has a bead concentration falling within 1×10⁹ to 4×10⁹/mL, and a solution capable of providing salt ions is an ammonium sulfate solution, with a final concentration falling within a range of 2 M to 3 M. In addition, in the step of adding a fetuin solution and a solution capable of providing salt ions into the bead solution, the culturing process takes place at 35° C. to 42° C. for 18 hours to 24 hours, and the content of fetuin-beads in the fetuin-bead solution falls within a range of 40 μg to 50 μg.

### Embodiment 2

### Method of Concentrating and Detecting Influenza Virus by Fetuin-Beads

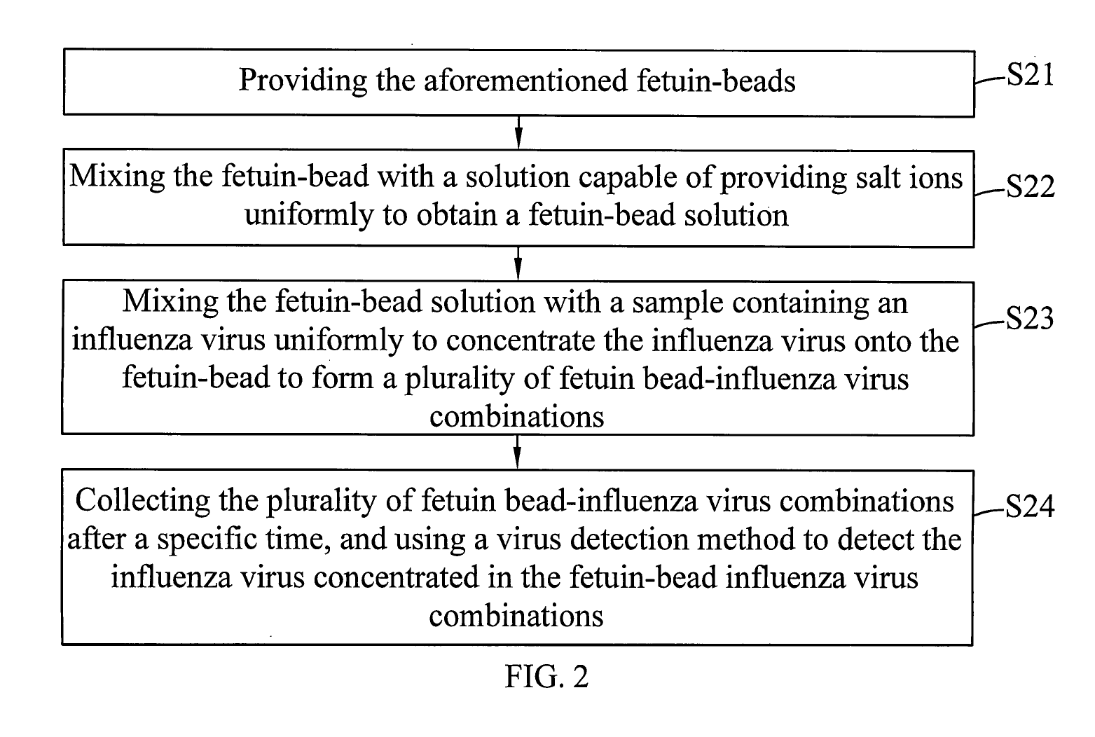

With reference to FIG. 2 for a flow chart of a method of concentrating and detecting influenza virus by the fetuin-bead with reference to the present invention, the detection method comprises the following steps. In Step S21, the aforementioned fetuin-beads are provided. In Step S22, the fetuin-beads are mixed uniformly with a solution capable of providing salt ions to obtain a fetuin-bead solution. In Step S23, the fetuin-bead solution is mixed uniformly with a sample of an influenza virus to concentrate the influenza virus onto the fetuin-bead to form a plurality of fetuin-bead influenza virus combinations. In Step S24, the fetuin-bead influenza virus combinations are collected after a specific time, and a virus detection method is used for detecting the influenza virus concentrated on the fetuin-bead influenza virus combinations.

Preferably, the aforementioned solution capable of providing salt ions is an ammonium sulfate solution, wherein the ammonium sulfate solution has a final concentration falling within a range 2 M to 3 M. In Step S23, the number of fetuin-beads in the fetuin-bead solution falls within a range of 2×10⁷ to 4×10⁷. The virus detection method includes but not limited to RNA extraction and real-time quantitative PCR, immunoblotting, hemagglutination inhibition, or any molecular biological method for examining influenza viruses.

### Embodiment 3

### Preferred Embodiment of the Present Invention

To make it easy for persons having ordinary skill in the art to understand the present invention, the following preferred embodiments and all of the following parameters and chemical test agents are provided for the purpose of illustrating the present invention, but not intended for limiting the scope of the present invention.

Materials of Experiment

Virus: human influenza virus H1N1 and a low pathogenic avian influenza virus H4N6 of which detailed information are listed in Table 1.

TABLE 1

| | Serial | Serial | Virus |
|---|---|---|---|
| | No. of Hemag- | No. of Neur- | Titration |
| Virus Strain | glutinin | aminidase | Concentration |
| A/Taiwan/70126/ | HM366455 | HM366456 | 4 × 10⁵ |
| 06/H1N1 | | | PFU/ml |
| A/Duck/Taiwan/ | HM366457 | HM366458 | 1.74 × 10⁴ |
| C59/2006(H4N6) | | | PFU/ml |

Bead: Dynabeads M-270 Epoxy (Model No.: Cat. No. 143.02D by Invitrogen).

Fetuin: Fetuin-F2379 (by SIGMA).

BSA (Bovine Serum Albumin)-SI-A7906-50G (by SIGMA).

Standard Protein: Prestained Protein Ladder Prep0925 (by Bioman).

Reverse Transcription Polymerase Chain (PCR) Reaction Primer and Real-time Quantitative Polymerase Chain Reaction Primers: Refer to Table 2.

TABLE 2

| Name of Primers | Sequence | Description |
|---|---|---|
| Uni-12 | 5′-AGC AAA AGC AGG-3′ | Transfer full-length |
| | | cDNA during reverse |
| | | transcription. |
| H1 504F | 5′-AGT TTT TAC ARA AAT | Clip out hemagglutinin |
| | TTG CTA TGG CTG AC-3′ | segment of H1N1. |
| H1 688R | 5′-GGG GTG AAT CTY CTG | Clip out hemagglutinin |
| | YTA TAA TKT CAA-3′ | segment of H1N1. |
| FluA-M52C | 5′-CTT CTA ACC GAG GTC | Clip out M segment of |
| | GAA ACG-3′ | H1N1. |
| FluA-M253R | 5′-AGG GCA TTT TGG ACA | Clip out M segment of |
| | AAK CGT CTA-3′ | H1N1. |
| M Fragment | 5′-(6-Fam)CCT CAA AGC | Diagnose M segment of |
| Probe | CGA GAT CGC GCA (Tamra)- | Influenza Virus A to |
| | 3′ | generate fluorescent |
| | | signal. |

Test Agents for Experiment

0.1 M sodium sulfate buffer solution: Prepare 2.62 g of sodium dihydrogen phosphate hydrate (NaH₂PO₄×H₂O with a molecular weight of 137.99) and 14.42 g of disodium hydrogen phosphate hydrate (Na₂HPO₄×2H₂O with a molecular weight of 177.99), add 800 mL of deionized distilled water (ddH₂O), adjust the pH value to 7.4, and then resupply the deionized distilled water (dd water) to 1 liter.

One time of phosphate buffer solution (PBS): Prepare and dissolve 8 g of sodium chloride, 0.2 g of potassium chloride, 1.44 g of disodium hydrogen phosphate and 0.24 g of potassium dihydrogen phosphate into 800 mL of deionized distilled water and stir uniformly by a magnetic rod, use 1 N HCl solution to adjust the pH value to 7.4, resupply the deionized distilled water to 1 liter, and finally sterilize the solution for future use.

6 M ammonium sulfate solution: Prepare and dissolve 7.93 g of ammonium sulfate ((NH₄)₂SO₄ with a molecular weight of 132.1) into 10 mL of 0.1M sodium phosphate buffer solution (pH 7.4). Filter the ammonium sulfate solution by a 0.22 nm membrane and package the solution for future use.

Phosphate solution containing 0.5% of milk: Dissolve 50 μg of skimmed milk powder into 10 mL of the phosphate buffer solution.

Sodium dodecyl sulfate polyacrylamide gel electrophoresis (SDS-PAGE): The composition of each agent is listed in Table 3.

TABLE 3

| | | | |
|---|---|---|---|
| | Total | | Total |
| Upper Stacking | Volume: | Lower Resolving | Volume: |
| Gel 5% | 5 ml | Gel 12% | 10 ml |

| | | | | | |
|---|---|---|---|---|---|
| deionized | 3.65 | ml | deionized | 4.3 | ml |
| distilled water | | | distilled water (dd | | |
| | | | water) | | |
| 40% acrylamide | 0.63 | ml | 40% acrylamide | 3 | ml |
| 1M Tris¹- | 0.63 | ml | 1.5M Tris- | 2.5 | ml |
| HCl pH 6.8 | | | HCl pH 8.8 | | |
| 10% SDS | 50 | μl | 10% SDS | 100 | μl |
| 10% APS² | 50 | μl | 10% APS | 100 | μl |
| TEMED³ | 5 | μl | TEMED | 10 | μl |

Note
¹Tris stands for Tris(hydroxymethyl)ammomethane.
Note
²APS stands for ammonium persulfate.
Note
³TEMED stands for tetramethyl ethylenediamine.

Manufacture of Fetuin-Beads

Firstly, the dry beads are added into 2 mL of diglyme to obtain a bead solution with a concentration of 2×10⁹/mL. Before use, the bead solution is shaken for 2 minutes, and then 300 μL of the bead solution is taken out. The bead solution contains 6×10⁸ beads. The bead solution is placed on the magnetic block for 4 minutes to remove the upper liquid.

600 μL of 0.1 M phosphate buffer solution is added into the bead solution and shaken for 30 seconds, mixed by a vibrator for 10 minutes, and then placed on a magnetic block for 4 minutes to remove the upper liquid. This step is repeated twice.

300 μL of 0.1 M phosphate buffer solution is added into the beads, such that every 50 μL of the bead solution contains 1×10⁸ beads. 50 μL of the bead solution is taken out and added with 25 μL of the fetuin solution with a concentration of 2 mg/mL, and then added with 75 μL of the ammonium sulfate solution with a concentration of 6 M, such that the ammonium sulfate solution has a final concentration of 3 M so as to obtain a mixed fetuin-bead solution. The mixed fetuin-bead solution is placed on the vibrator and cultured at 37° C. for 24 hours (overnight). Next day, the mixed fetuin-bead solution is centrifuged at the magnetic block for 2 minutes to remove the upper liquid. 400 mL of 0.1 M sodium sulfate buffer solution is rinsed once and shaken for 10 seconds each time, and then placed on the magnetic block for 2 minutes to remove the upper liquid.

200 μL of phosphate solution containing 0.5% milk is added to obtain a fetuin-bead solution. The fetuin-bead solution is shaken for 10 seconds and then stored overnight at 4° C. to obtain a fetuin-bead. The fetuin-bead in the fetuin-bead solution is kept at a temperature below −20° C. Before using the fetuin-beads of the fetuin-bead solution, 400 μL of the phosphate buffer solution is used for rinsing and then removed. Finally, 150 μL of the phosphate buffer solution is added, so that every 30 μL of the fetuin-bead solution contains 2×10⁷ fetuin-beads.

Concentrating Viruses in Water by Fetuin-Beads

20 μL of human influenza virus H1N1 solution is prepared as a virus load, and then added into 980 μL of dd water to produce two control groups of human influenza virus H1N1, called as a first control group and a second control group, respectively. 20 μL of the first control group is taken out to use as a virus dilution control group. 4×10⁷ fetuin-beads (i.e. 60 μL of fetuin-bead Milk solution) are added into the second control group, placed on the vibrator and shaken at room temperature for 1 hour. Then, the fetuin-beads are taken out from the second control group and rinsed by 200 μL of the phosphate buffer solution twice to recover the fetuin-beads. The recovered fetuin-beads, 20 μL of two control groups and 20 μL of the virus dilution control group are added with deionized distilled water, respectively to 140 μL. A QIAamp Viral RNA Mini Kit (Model No.: Cat. No. 52904) is used for extracting a human influenza virus RNA, and a reverse transcription of the human influenza virus RNA is performed by a two-step RT-PCR system to obtain a human influenza virus cDNA. A SuperScript III Platinum® One-Step Quantitative RT-PCR System (Model No.: Cat. No. 11732-088) is used for a real-time quantitative PCR of the human influenza virus cDNA.

Limitations of Detecting and Measuring Fetuin-Beads

In a test of the detection limit of the fetuin-beads, a phosphate buffer solution is used for performing a 10× serial dilution to dilute the human influenza virus H1N1 (4×10⁵ PFU/mL) into 4000, 400, 40, 4, and 0.4 PFU/mL. Additionally, a phosphate buffer solution is used to dilute a low pathogenic avian influenza virus H4N6 (1.74×10⁴ PFU/mL) into 1740, 174, 17.4, 1.74, and 0.174 PFU/mL, and the final volume is equal to 1080 μL. Each diluted solution of the two virus strains (human influenza virus H1N1 and low pathogenic avian influenza virus H₄N₆) is taken out 280 μL to divide into two groups of the virus solution with a volume of 140 μL. One group serves as a non-concentrated virus group, and another group serves as a concentrated fetuin-bead group. In the concentrated fetuin-bead group, the fetuin-beads are used for concentrating viruses, and the fetuin-beads are recovered after the concentration is completed. After the fetuin-beads is redissolved by 140 μL of the phosphate buffer solution, RNAs of the human influenza virus H1N1 and low pathogenic avian influenza virus H4N6 are extracted, and then a real-time quantitative PCR of M segments is performed.

Reinfection Assay of Embryonated Chicken Eggs

In a virus infection test, a conventional embryonated chicken egg amplification method is adopted. An embryonated chicken egg with 9-day old specific pathogen free (SPF) is prepared. A 10× serial dilution of the virus sample to be detected is performed by using the phosphate buffer solution containing 1% of antibiotic. A serial dilution of the human influenza virus H1N1 (4×105 PFU/ml) is perform to dilute the concentrations to 400, 40, 4, and 0.4 PFU/mL. A low pathogenic avian influenza virus H4N6 (1.74×104 PFU/ml) is diluted separately into concentrations of 17.4, 1.74, 0.174, and 0.0174 PFU/mL. The final volume of each diluted solution of the aforementioned human influenza virus H1N1 and low pathogenic avian influenza virus H4N6 is equal to 1080 μL. Each diluted solution of the two virus strains (human influenza virus H1N1 and low pathogenic avian influenza virus H₄N₆) is manufactured to produce two groups of virus solution, wherein one group serves as a non-concentrated virus group, and another group serves as a concentrated fetuin-bead group. Each diluted virus solution of the non-concentrated virus group is inoculated into five chicken eggs separately, and 100 μL of the solution is injected into each egg. In another group serving as the concentrated fetuin-bead group, the viruses are concentrated by the fetuin-beads, and the fetuin-beads concentrated with the viruses are recovered. After each recovered fetuin-beads is redissolved by the phosphate buffer solution containing 1% of antibiotic, each diluted solution is inoculated into two eggs, and 100 μL of the solution is inoculated into each egg. An allantoic fluid of the embryonated chicken egg is collected after the post-inoculation for 24 hours. RNAs of the human influenza virus H1N1 and low pathogenic avian influenza virus H4N6 in each allantoic fluid are extracted, and then a real-time quantitative PCR of the M segment is performed.

Analysis on Recovery Rate of Human Influenza Virus H1N1

The CP (crossing point) value of the real-time quantitative PCR of the aforementioned virus stock solution control group subtracts the CP value of the fetuin-bead to obtain ΔCP, and the ΔCP is converted into two squares of ΔCP, so as to obtain a human influenza virus recovery rate of being concentrated by the fetuin-bead.

### Experiment Result

1. Preparing and Testing Fetuin-Beads

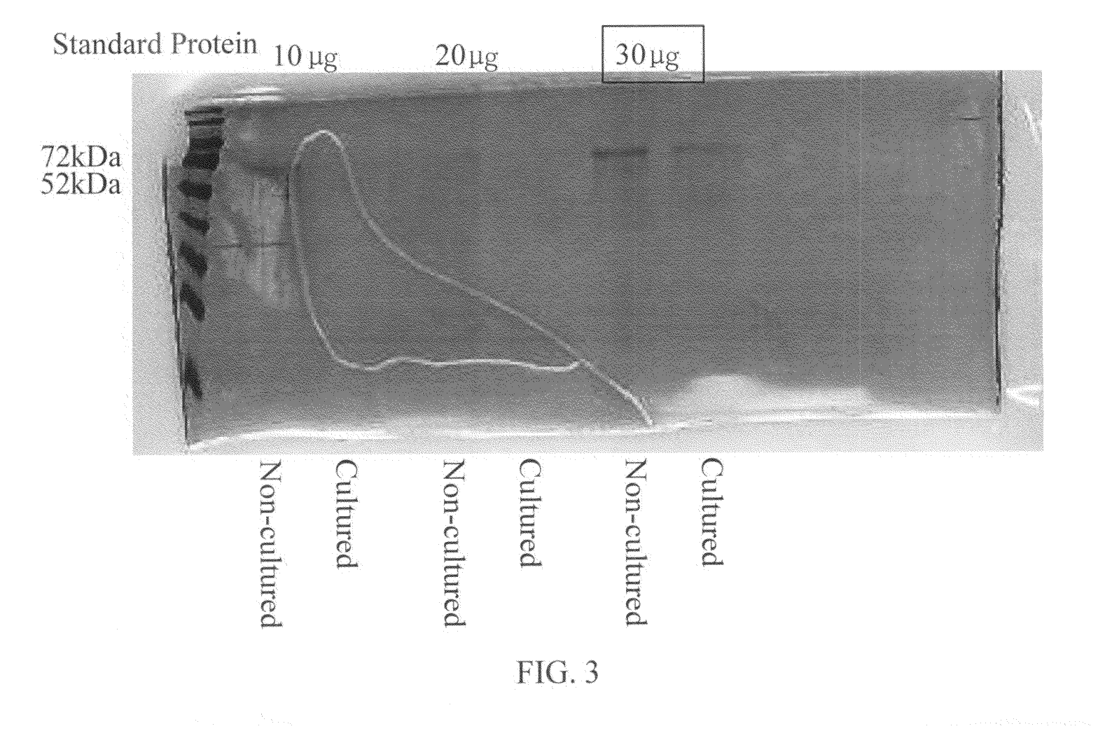

To confirm if the fetuin can be combined with the beads well, three groups of bead solution each having 50 μL (1×10⁸) mixed separately with 2.5 μL, 5 μL, and 15 μL (2 mg/mL) of fetuin and added with 75 μl, of the 6 M ammonium sulfate solution to help combining the fetuin with the beads. Then, a phosphate buffer solution is provided to make up the solution to 150 μL and mixed uniformly, and the ammonium sulfate solution has a final concentration of 3 M. Subsequently, 20 μL of the fetuin-bead solution is added with 5×SDS staining solution, frozen and stored at −20° C., and the remaining 130 μL of the bead solution is placed on the vibrator and cultured at 37° C. for 16 hours. In the next day, the fetuin-bead solution is taken out and centrifuged, and 20 μL of the upper liquid is taken out and added with a 5×SDS staining solution. Such solution and 20 μL of the fetuin-bead solution pre-frozen at −20° C. in the previous day are heated together at 99° C. for 10 minutes. After heating 10 minutes, the SDS-PAGE is performed. The results are shown in FIG. 3.

In FIG. 3, when the bead solution is added into 2.5 μL and 5 μL of the fetuin, no band detected from the upper liquid of the cultured fetuin-bead solution is observed. It indicates that the fetuin has combined completely with the beads, but the combination is not saturated yet. When the bead solution is added into 15 μL of the fetuin, light colored bands detected from the upper liquid of the cultured fetuin-bead solution is observed. Although it is uncertain that the combination of the fetuin and the beads is saturated in this condition, the result shows that the fetuin is combined with the beads already.

2. Virus Recovery Rate of Fetuin-Beads

After confirming the combination of the fetuin with the beads, the ability for the fetuin-bead to be absorbed with the influenza virus particles is detected. The fetuin-bead (4×10⁷) of a 3 M ammonium sulfate solution is added into 1 mL of the non-concentrated virus solution with the concentration of 8000 PFU/mL (which is the virus dilution control group), and then placed on the vibrator at room temperature and shaken for 1 hour. The fetuin-bead is taken out, and rinsed by 200 μL of the phosphate buffer solution twice. RNAs of the recovered fetuin-bead, the 20 μL of the upper liquid of the recovered fetuin-beads, 20 μL of the virus stock solution of the first and second control groups, and 20 μL of the virus dilution control group are extracted, and then a reverse transcription of the RNAs is performed to obtain a human influenza virus cDNA. A real-time quantitative PCR of the cDNA is performed to obtain an average CP value of 24.14 in a virus stock solution of the first and second control groups (i.e. the control group of human influenza virus H1N1). 1 mL of the virus dilution control group has an average CP value of 28.97; the recovered fetuin-bead has an average CP value of 25.61; and the upper liquid of the fetuin-bead solution has an average CP value of 31.35. The recovery rate of the virus stock solution of the first and second control groups serves as a basis of 100% for converting the recovery rate of each group, and the results are listed in Table 4.

TABLE 4

| | | ΔCP = CP value | | |
|---|---|---|---|---|
| | | of virus stock | | |
| | Aver- | solution − CP | | |
| | age CP | value of | | Recovery |
| | Value | each group | 2\hat{ }ΔCP | Rate |
| Virus stock | 24.14 | 0 | 1 | 100% |
| solution | | | | |
| Virus dilution | 28.97 | −4.83 | 0.035158 | 3.50% |
| control group | | | | |
| Recovered fetuin- | 25.61 | −1.47 | 0.360982 | 36% |
| beads | | | | |
| Upper liquid of | 31.35 | −7.21 | 0.006754 | 0.67% |
| fetuin-bead | | | | |
| solution | | | | |

The results show that the recovery rate of the virus dilution control group is 3.50% which is close to 2.0% of the expected value thereof; the recovered fetuin-bead has a recovery rate of 36%; and the upper liquid of the fetuin-bead solution has a recovery rate of 0.67%. It indicates that the virus concentration of the upper liquid of the fetuin bead solution is significantly lower than that of the virus dilution control group. Thus, the results prove that the fetuin-beads actually can adsorb the virus particles in water.

3. Optimal Condition for Manufacturing Fetuin-Beads

The test further takes place at different experimental conditions to find out the best condition for combining the fetuin and beads according to the effect of fetuin adsorbed on the surface of the beads.

(1) Concentration of Ammonium Sulfate Solution

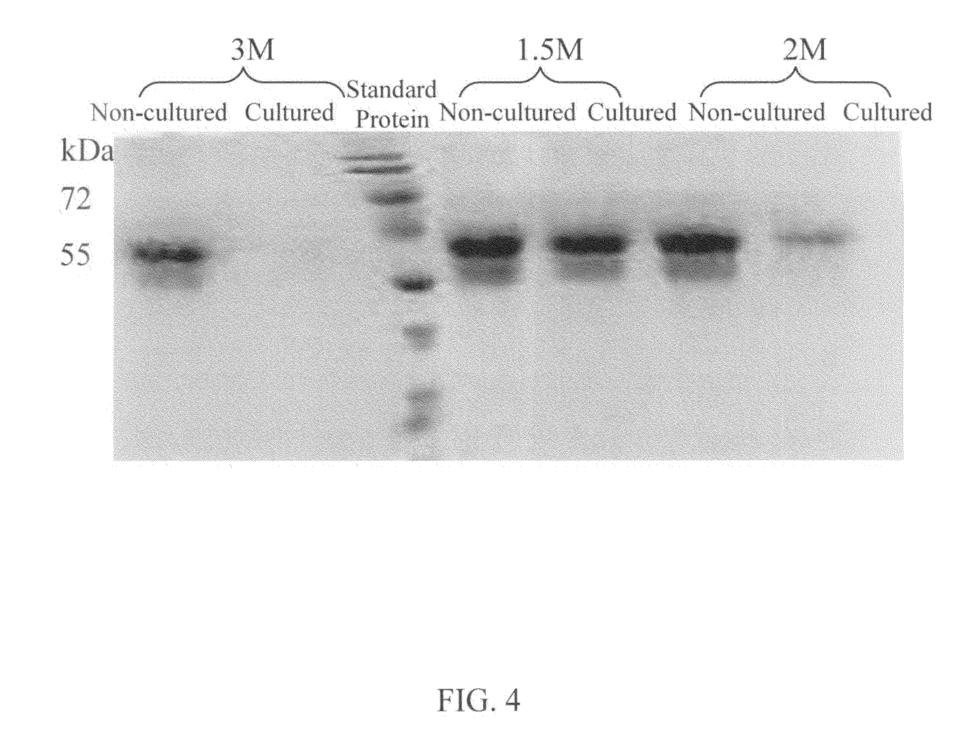

In three groups of bead solutions, 50 μL (1×10⁸) of each group is mixed with 20 μL (2 mg/mL) of the fetuin solution, and different volumes of 75 μL, 50 μL and 37.5 μL of 6 M ammonium sulfate solution are added into the three groups respectively. The phosphate buffer solution is added into the remaining solution until the total volume is 150 μL, such that the final concentrations of the ammonium sulfate solution are 3 M, 2 M and 1 M, respectively. Then, a 5× concentration SDS staining solution is added into 20 μL of each group of the bead solutions, frozen at −20° C., and the remaining 130 μL of the solution is placed on the vibrator and cultured at 37° C. for 18 hours. In the next day, the solution with the volume of 130 μL is centrifuged, and 20 μL of the upper liquid is taken out and added with 5× concentration SDS staining solution. Such solution and 20 μL of the solution pre-frozen at −20° C. in the previous day are heated together at 99° C. for 10 minutes, and then a SDS-PAGE electrophoresis is performed, and the results are shown in FIG. 4.

With reference to FIG. 4, for an ammonium sulfate solution group having the concentration of 1.5 M, the intensity of non-cultured and cultured bands remains unchanged. The intensity of the bands after the cultured bands is changed significantly to a lighter color in the ammonium sulfate solution group having the concentration of 2 M; and the intensity of the cultured bands is changed to a very light color or almost invisible in the ammonium sulfate solution group having the concentration of 3 M. These results show that the best concentration of the ammonium sulfate solution is 3M.

(2) Temperature

Because heating will open up the structure of the protein to expose more binding sites, a test is carried out to determine whether the heated fetuin can combine with the beads more easily or not.

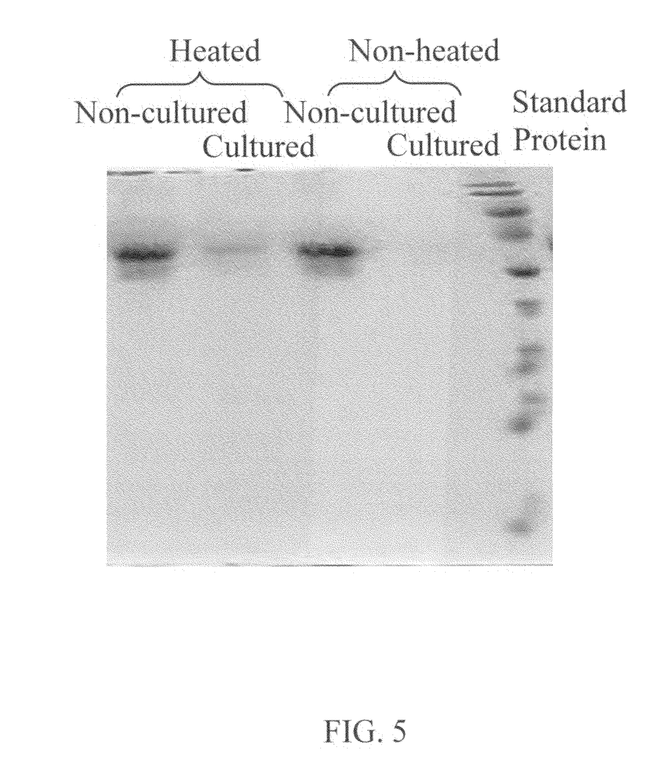

Firstly, two groups of bead solutions, each having the volume of 50 μL (or 1×10⁸ beads), are prepared. One group is added with 20 μL (2 mg/mL) of a non-heated fetuin solution, and another group is added with 20 μL (2 mg/mL) of a fetuin solution preheated at 99° C. for 10 minutes. In each group, 75 μL of 6 M ammonium sulfate solution is added, and then a phosphate buffer solution is added until the volume is 150 μL, such that the final concentration of the ammonium sulfate solution is 3 M. A 5× concentration SDS staining solution is added into 20 μL of the solution of each group, and frozen at −20° C. The remaining 130 μL of the solution is placed on the vibrator and cultured at 37° C. for 18 hours. In the next day, the solution is taken out and centrifuged, and then 20 μL of the upper liquid is collected, and added with the 5× concentration SDS staining solution. Such solution and the 20 μL of the solution pre-frozen at −20° C. in the previous day are heated at 99° C. for 10 minutes, and then the SDS-PAGE electrophoresis is performed. The results are shown in FIG. 5.

With reference to FIG. 5, the results show that the bands of the heated fetuin are more obvious than the bands of non-heated fetuin, regardless of being cultured or not cultured. The result indicates that the effect of combining a heated fetuin with the beads drops instead.

(3) Culturing Time

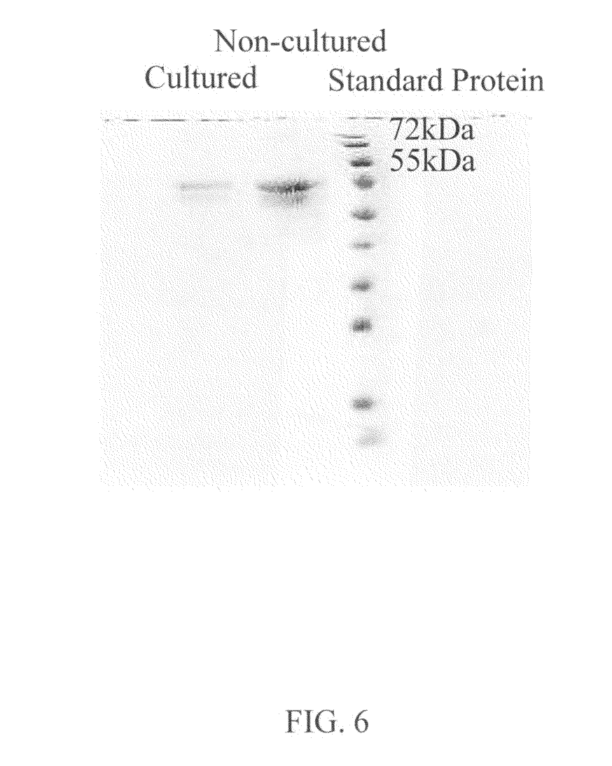

50 μL (1×10⁸ beads) of the bead solution is mixed with 25 μL (2 mg/mL) of the fetuin solution, and 75 μL of 6 M ammonium sulfate solution is added to help combining the fetuin with the beads, such that the total volume is 150 μL and the final concentration of the ammonium sulfate solution is 3 M. 20 μL of the fetuin-bead solution is prepared and added with the 5× concentration SDS staining solution, and then frozen at −20° C. The remaining 130 μL of the fetuin-bead solution is placed on the vibrator and cultured at 37° C. for 24 hours according to the recommended maximum culturing time of manufacturing the fetuin-beads. In the next day, 130 μL of the cultured fetuin-bead solution is taken out and centrifuged, and then 20 μL of the upper liquid is taken out and added with the 5× concentration SDS staining solution. Such solution and 20 μL of the fetuin-bead solution pre-frozen at −20° C. in the previous day are heated at 99° C. for 10 minutes, and then the SDS-PAGE Electrophoresis test is performed. The results are shown in FIG. 6.

With reference to FIG. 6, the bands detected from the upper liquid of the fetuin cultured for 24 hours still can be observed, indicating that the binding of the fetuin with the beads is saturated.

The experimental results of the aforementioned three groups show that the best way of manufacturing the fetuin-beads is by adding 50 μL of the bead solution (containing 1×10⁸ beads) and 25 μL of the fetuin solution (with a concentration of 2 mg/mL), and then adding 75 μL of the 6 M ammonium sulfate solution, and culturing the solution at 37° C. for 24 hours. The following fetuin-beads are manufactured by above conditions.

4. Influenza Virus Recovery Rate at Different Quantities of Fetuin-Beads

The influenza virus recovery rates of the three groups of fetuin-beads manufactured above are tested to find the influenza virus recovery rate of the fetuin-beads manufactured according to the best condition.

4×10⁷, 2×10⁷, 1×10⁷, 5×10⁶ and 1.3×10⁶ fetuin-beads manufactured according the optimal condition are separately added with 1 mL of virus dilution solution containing 20 μL of human influenza viruses, and the solutions are shaken for 1 hour. The fetuin-beads are taken out and rinsed by 200 μL of the phosphate buffer solution twice. RNAs are extracted from all recovered fetuin-beads, 20 μL of the virus stock solution and 20 μL of the non-concentrated virus dilution solution. A reverse transcription of the RNAs is performed, and a quantitative PCR of the cDNAs is performed. The results are listed in Table 5 as follows.

TABLE 5

| | | ΔCP = CP value | | |
|---|---|---|---|---|
| | | of virus stock | | |
| | Aver- | solution − CP | | |
| | age CP | value of | | Recovery |
| | Value | each group | 2\hat{ }ΔCP | Rate |
| Virus stock | 23.435 | 0 | 1 | 100% |
| solution | | | | |
| Virus diluted | 29.045 | −5.61 | 0.020475 | 2.04% |
| Solution | | | | |
| 4 × 10⁷ beads | 23.3275 | 0.1075 | 1.07736 | 107.74% |
| 2 × 10⁷ beads | 23.3625 | 0.0725 | 1.051537 | 105% |
| 1 × 10⁷ beads | 23.53 | −0.095 | 0.936272 | 94% |
| 5 × 10⁶ beads | 24.7025 | −1.2675 | 0.415379 | 42% |
| 1.3 × 10⁶ beads | 26.6775 | −3.2425 | 0.10566 | 11% |

Wherein, the virus stock solution has an average CP value of 23.43, and the virus dilution solution has an average CP value of 29.04. The average CP values are obtained by repeating the groups with different quantities of beads. The group with 4×10⁷ beads has an average CP value of 23.32; the group with 2×10⁷ beads has an average CP value of 23.36; the group with 1×10⁷ beads has an average CP value of 23.53; the group with 5×10⁶ beads has an average CP value of 25.05; and the group with 1.3×10⁶ beads has an average CP value of 26.82.

Based on the CP value of the virus stock solution converted into the recovery rate as 100%, the proportion of RNA concentration in each group is converted into the recovery rate. The 50× diluted virus dilution solution has a recovery rate of 2.04% which is very close to the expected value of 2.0%. The group with 4×10⁷ beads has a recovery rate of 107.74%. The group with 2×10⁷ beads has a recovery rate of 105%. The group with 1×10⁷ beads has a recovery rate of 94%. The group with 5×10⁶ beads has a recovery rate of 42%, and the group with 1.3×10⁶ beads has a recovery rate of only 11%.

The foregoing results show that the recovery rate for the group with 1×10⁷ fetuin-beads or less has a significant drop, and it can deduce that the concentration of 1×10⁷ fetuin-beads gives a better recovery rate. Although the recovery rate for the concentrations of 4×10⁷ and 2×10⁷ beads exceeds 100%, the recovery rate is very close to 100% within the range of acceptable errors. The results also confirm that the best manufacturing condition of the fetuin-beads is to culture 25 μL of the fetuin solution (with a concentration of 2 mg/mL) at 37° C. for 24 hours and use the ammonium sulfate solution with a concentration of 3 M.

5. Limitation of Detection and Reinfection of Fetuin-Beads

A 10× serial dilution of the human influenza virus H1N1 and the low pathogenic avian influenza virus H4N6 solutions are performed, and the fetuin-beads with the same concentration (2×10⁷ beads) are used for concentrating the aforementioned serial dilution virus solution. A real-time quantitative PCR and an embryonated chicken egg infection test are performed. The results of the real-time quantitative PCR are listed in Table 6 as follows.

TABLE 6

| | Testing Result | | | | |
|---|---|---|---|---|---|
| | (Final virus concentration, unit in PFU/mL) | | | | |
| Human | 0.4 | 4 | 40 | 400 | 4000 |
| influenza | | | | | |
| virus H1N1 | | | | | |
| Non- | Nil | Nil | 2/2 | 2/2 | 2/2 |
| concentrated | | | | | |
| fetuin-beads | | | | | |
| Concentrated | 1/2 | 2/2 | 2/2 | 2/2 | 2/2 |
| fetuin-beads | | | | | |
| Low | 0.172 | 1.72 | 17.2 | 172 | 1720 |
| pathogenic | | | | | |
| avian | | | | | |
| influenza | | | | | |
| virus H4N6 | | | | | |
| Non- | Nil | 2/2 | 2/2 | 2/2 | 2/2 |
| concentrated | | | | | |
| fetuin-beads | | | | | |
| Concentrated | 1/2 | 2/2 | 2/2 | 2/2 | 2/2 |
| fetuin-beads | | | | | |

Note 1:
“Nil” refers to no influenza virus can be detected from two repeated testing results.
Note 2:
“1/2” refers to the influenza virus can be detected for only one out of two repeated testing results.
Note 3:
“2/2” refers to the influenza virus can be detected two out of two repeated testing results.

The above results show that the influenza virus solution concentrated by the fetuin-beads has a concentration equal to 10 to 100 times of the concentration of the non-concentrated fetuin-beads within the detection limit, and the fetuin-bead has a very good virus concentration effect.

In addition, the results of the embryonated chicken egg infection test are listed in Table 7 as follows.

TABLE 7

| | Testing Result | | | |
|---|---|---|---|---|
| | (Final virus concentration, unit in PFU/mL) | | | |
| Human | 0.4 | 4 | 40 | 400 |
| influenza | | | | |
| virus H1N1 | | | | |
| Non- | 0/3* (40%) | 2/5 (40%) | 2/2* (100%) | 3/3* (100%) |
| concentrated | | | | |
| fetuin-beads | | | | |
| Concentrated | 4/4* (100%) | 4/5 (80%) | 4/5 (80%) | 4/4* (100%) |
| fetuin-beads | | | | |
| Low | 0.172 | 1.72 | 17.2 | 172 |
| pathogenic | | | | |
| avian | | | | |
| influenza | | | | |
| virus H4N6 | | | | |
| Non- | 2/5 (40%) | 4/5 (80%) | 4/4* (100%) | 3/3* (100%) |
| concentrated | | | | |
| fetuin-beads | | | | |
| Concentrated | 4/5* (80%) | 5/5* (100%) | 4/4* (100%) | 3/3* (100%) |
| fetuin-beads | | | | |

Note 1:
“Nil” refers to no influenza virus can be detected from two repeated testing results.
Note 2:
“1/2” refers to the influenza virus can be detected for only one out of two repeated testing results.
Note 3:
“2/2” refers to the influenza virus can be detected two out of two repeated testing results.

The above results show that the influenza virus adsorbed by the fetuin-bead still has still a reinfection effect.

In summation, all of the foregoing tests adopt the fetuin-beads of the present invention to detect and separate an influenza virus, and the fetuin-beads can be used extensively in hospitals or research institutions, particularly for the applications on monitoring influenza viruses at early-stage identifications and preventing new influenzas. In addition, the influenza virus concentrated by the fetuin-beads of the present invention still has the ability of infecting chicken embryos, and this feature can be applied to subsequent subcultures including research of viruses to improve the clinical and future molecular biological analysis substantially.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A manufacturing method of a fetuin-bead, comprising:
preparing a bead solution including a plurality of beads; and
adding a fetuin solution and a solution capable of providing salt ions into the bead solution and performing a shaking process and a culturing process to obtain a fetuin-bead solution containing a plurality of fetuin-beads.

2. The manufacturing method of a fetuin-bead as recited in claim 1, wherein a concentration of the plurality of beads in the bead solution is in a range of 1×10⁹ to 4×10⁹/mL.

3. The manufacturing method of a fetuin-bead as recited in claim 1, wherein the solution capable of providing salt ions is an ammonium sulfate solution.

4. The manufacturing method of a fetuin-bead as recited in claim 3, wherein the ammonium sulfate solution has a final concentration falling within a range of 2 M to 3 M.

5. The manufacturing method of a fetuin-bead as recited in claim 1, wherein in the step of adding the fetuin solution and the solution capable of providing salt ions into the bead solution, the culturing process is taken place at 35° C. to 42° C. for 18 hours to 24 hours.

6. The manufacturing method of a fetuin-bead as recited in claim 1, wherein the fetuin-bead solution has a content of fetuin-beads falling within a range of 40 to 50 μg.

7. A fetuin-bead, manufactured by the manufacturing method of a fetuin-bead as recited in claim 1.

8. The fetuin-bead as recited in claim 7, wherein a concentration of the plurality of beads in the bead solution is in a range of 1×10⁹ to 4×10⁹/mL.

9. The fetuin-bead as recited in claim 7, wherein the solution capable of providing salt ions is an ammonium sulfate solution.

10. The fetuin-bead as recited in claim 9, wherein the ammonium sulfate solution has a final concentration falling within a range of 2 M to 3 M.

11. The fetuin-bead as recited in claim 7, wherein in the step of adding the fetuin solution and the solution capable of providing salt ions into the bead solution, the culturing process is taken place at 35° C. to 42° C. for 18 hours to 24 hours.

12. The fetuin-bead as recited in claim 7, wherein the fetuin-bead solution has a content of fetuin-beads falling within a range of 40 to 50 μg.

13. A method of concentrating and detecting influenza virus by the fetuin-beads, comprising the steps of:
providing the fetuin-bead as recited in claim 7;
mixing the fetuin-bead with a solution capable of providing salt ions uniformly to obtain a fetuin-bead solution;
mixing the fetuin-bead solution with a sample containing an influenza virus uniformly to concentrate the influenza virus onto the fetuin-bead to form a plurality of fetuin bead-influenza virus combinations; and
collecting the plurality of fetuin bead-influenza virus combinations after a specific time, and using a virus detection method to detect the influenza virus concentrated in the fetuin-bead influenza virus combinations.

14. The method of concentrating and detecting influenza virus by the fetuin-beads as recited in claim 13, wherein the solution capable of providing salt ions is an ammonium sulfate solution.

15. The method of concentrating and detecting influenza virus by the fetuin-beads as recited in claim 14, wherein the ammonium sulfate solution has a final concentration falling within a range of 2 M to 3 M.

16. The method of concentrating and detecting influenza virus by the fetuin-beads as recited in claim 13, wherein the fetuin-bead solution used in the step of mixing the fetuin-bead solution and the sample containing the influenza virus has 2×10⁷ to 4×10⁷ fetuin-beads.

17. The method of concentrating and detecting influenza virus by the fetuin-beads as recited in claim 13, wherein the virus detection method is one selected from RNA extraction and real-time quantitative PCR, immunoblotting, and hemagglutination inhibition.
